(19) United States
(12) Patent Application Publication
(10) Pub. No.: US 2026/0294997 A1
(43) Pub. Date: Oct. 1, 2026

(54) ZINGIBER ZERUMBET EXTRACT AND PROCESS OF MAKING AND USING THE SAME

(71) Applicant: TAIPEI MEDICAL UNIVERSITY, Taipei City (TW)

(72) Inventors: Ching-Chiung WANG, Taipei City (TW); Wen-Chi HOU, Taipei City (TW)

(21) Appl. No.: 19/095,340

(22) Filed: Mar. 31, 2025

(51) Int. Cl. A61K 36/9068; A61K 31/015; A61K 31/045; A61K 31/122; A61K 31/336; A61K 31/352; A61P 19/02; A61P 25/28

(52) U.S. Cl. CPC A61K 36/9068; A61K 31/015; A61K 31/045; A61K 31/122; A61K 31/336; A61K 31/352; A61P 19/02; A61P 25/28; A61K 2236/37

(57) ABSTRACT

The present disclosure relates to rhizome extract, extract composites, and active component of Taiwan zerumbet ginger (Zingiber zerumbet), and the process of making the same. The present disclosure also relates to methods of administering the rhizome extract, extract composites, and active component of Zingiber zerumbet to a subject for improving cognitive dysfunctions in neurodegenerative diseases or for treating osteoarthritis.

### TECHNICAL FIELD OF THE INVENTION

The present disclosure relates to a field of herbal product and its applications. Particularly, the present disclosure provides a rhizome extract, extract composites, and the active component of Taiwan zerumbet ginger (Zingiber zerumbet), process of making the same and its applications in improving cognitive dysfunctions arising from neurodegenerative diseases, preventing and/or treating osteoarthritis in a subject.

### BACKGROUND OF THE INVENTION

According to the systematic analysis report of the Global Burden of Disease (GBD) Study in 2019, among adults aged from 50 to 74, low back pain ranks 8th, and osteoarthritis ranks 24th in the leading causes of disease burden worldwide; Alzheimer's disease and dementia do not rank in the top 25. However, among adults aged 75 and older, Alzheimer's disease and dementia rank 4th, while low back pain ranks 13th and osteoarthritis ranks 22nd in the leading causes of disease burden worldwide. The results indicate that in a super-aged society, low back pain, osteoarthritis, and cognitive decline diseases are all significant factors affecting quality of life.

Research shows that patients suffering from Alzheimer's disease begin to experience cerebral changes or neuronal damage twenty years before memory loss occurs or even earlier. The number of damaged neurons and the areas of the brain affected determine the progression of Alzheimer's disease.

There is thus a need for compositions and methods for prevention and treatment of the diseases as mentioned above.

### SUMMARY OF THE INVENTION

The inventors intend to develop new and efficient extraction for Zingiber zerumbet to provide extracts and related products having sufficient amounts of zerumbone and other active components to exhibit the desired biological activities and higher bioavailability, and substantiate the effectiveness of these methods with evidence based on pharmacological data (particularly from serum) and pharmacokinetic data.

The present disclosure thus relates to a Zingiber zerumbet rhizome extract, characterized in comprising:
(a) at least 500 mg/g zerumbone/extract
(b) α-humulene,
(c) caryophyllene oxide, and
(d) β-eudesmol.

In one embodiment, the extract further comprises:
(e) camphene, and
(f) eucalyptol.

The present disclosure also relates to a Zingiber zerumbet rhizome extract composite, characterized in comprising at least 120 mg/g zerumbone/extract and at least 5.0 mg/g 3-O-methyl kaempferol/extract.

In one embodiment, the extract composite further comprises:
(b) α-humulene,
(c) caryophyllene oxide, and
(d) β-eudesmol.

In any preceding embodiment, the extract further comprises:
(e) camphene, and
(f) eucalyptol.

The present disclosure further relates to a process of making Zingiber zerumbet rhizome extract, comprising conducting extraction of Zingiber zerumbet with supercritical fluid carbon dioxide (CO₂) or via steam distillation.

In any preceding aspects or embodiments, the Zingiber zerumbet rhizome extract or extract composite is obtained by the process of making the same as described herein.

The present disclosure further relates to a process of making Zingiber zerumbet rhizome extract composite, comprising:
(a) conducting extraction of Zingiber zerumbet with supercritical fluid carbon dioxide (CO₂) to give a first extract and a first residue;
(b) conducting extraction of the first residue obtained in step (a) with an ethanolic solution to give a second extract and a second solid residue;
(c) combining the first extract obtained in step (a) and the second extract obtained in step (b) in a weight ratio of 1:9.5 to 1:1.2 to give the Zingiber zerumbet rhizome extract composite.

The present disclosure further relates to a process of making Zingiber zerumbet rhizome extract composite, comprising:
(a) conducting extraction of Zingiber zerumbet via steam distillation to give a first extract and a first residue;
(b) conducting extraction of the first solid residue obtained in step (a) with an ethanolic solution to give a second extract and a second residue;
(c) combining the first extract obtained in step (a) and the second extract obtained in step (b) in a weight ratio of 1:9.5 to 1:1.2 to give the Zingiber zerumbet rhizome extract composite.

The present disclosure further relates to a Zingiber zerumbet rhizome extract composite obtained by the method as described herein.

In any preceding aspects or embodiments, the Zingiber zerumbet rhizome extract composite is obtained by combining two or more of the Zingiber zerumbet rhizome extracts.

The present disclosure also relates to a pharmaceutical composition comprising the Zingiber zerumbet rhizome extract or extract composite as described herein.

The present disclosure also relates to a method of improving cognitive dysfunctions in neurodegenerative diseases in a subject, comprising administering an effective amount of the Zingiber zerumbet rhizome extract or extract composite as described herein to the subject.

The present disclosure also relates to a pharmaceutical composition for improving cognitive dysfunctions in neurodegenerative diseases, comprising the Zingiber zerumbet rhizome extract or extract composite as described herein, and a pharmaceutically acceptable carrier.

The present disclosure also relates to a method of preventing and/or treating osteoarthritis in a subject, comprising administering an effective amount of the Zingiber zerumbet rhizome extract or extract composite as described herein to the subject.

The present disclosure also relates to a pharmaceutical composition for preventing and/or treating osteoarthritis, comprising the Zingiber zerumbet rhizome extract composite as described herein, and a pharmaceutically acceptable carrier.

The present disclosure also relates to a food additive, a food product and/or a healthcare food product for joint health maintenance or neuroprotection, comprising the Zingiber zerumbet rhizome extract or extract composite as described herein.

In any preceding aspects or embodiments, the osteoarthritis is primary osteoarthritis, occupational osteoarthritis and/or secondary osteoarthritis.

### BRIEF DESCRIPTION OF THE DRAWINGS

FIGS. 1A and 1B show the neurotrophic factor activity of the claimed extracts for promoting the growth of PC12 cell neurites.

FIGS. 2A and 2B show neuroprotection effects of the claimed extracts.

FIGS. 3A and 3B show the protocol and test results for improvements of cognitive dysfunctions in an animal model.

FIG. 4 shows the protocol and test results for delaying aging, improving oxidative stress and learning ability in an animal model.

FIGS. 5A and 5B show the H&E staining on brain sections of mice in the galactose-induced oxidative stress aging mice model.

FIG. 6 shows the DPPH free radical scavenging assay results.

FIGS. 7A to 7C show the variation in PGE₂ concentration, protein expression of ADAMTS4 and aggrecan in cartilage and knee widths upon feeding with Extract I or Extract J observed in an animal model.

FIG. 8 shows H&E staining, Safranin O staining and Toluidine blue staining on femoral joint tissues of rats in different groups.

### DETAILED DESCRIPTION OF THE INVENTION

In order to facilitate understanding of the disclosure herein, terms as used herein are defined below.

In the context of the specification and the claims, the singular forms “a,” “an,” and “the” include plural referents, unless specifically indicated otherwise. Unless otherwise stated, any and all examples exemplary language (e.g., “such as”) provided herein are merely used for better illustration of the present invention, instead of limiting the scope of the present invention.

It is to be understood that any numerical range recited in this specification is intended to include all sub-ranges encompassed therein. For example, a range from “50 to 70° C.” includes all sub-ranges and specific values between the stated minimum value of 50° C. and the stated maximum value of 70° C., inclusive, e.g., from 58° C. to 67° C., and from 53° C. to 62° C., 60° C. or 68° C. Since the numerical ranges disclosed are continuous, they contain each numerical value between the minimum and maximum value. Unless otherwise specified, the various numerical ranges indicated in this specification are approximate.

In the present invention, the term “about” refers to an acceptable deviation of a given value measured by a person of ordinary skill in the art, depending, in part, on how the value is measured or determined. For example, the term “about” may refer to +/−10% differences of the indicated value.

The term “subject” includes living organisms such as humans, monkeys, cows, sheep, horses, pigs, cattle, goats, dogs, cats, mice, rats, cultured cells, and transgenic species thereof. In a preferred embodiment, the subject is a human.

The term “administering” includes routes of administration which allow the active ingredients of the present disclosure to perform their intended function.

The term “treat” or “treatment” refers to a method of reducing the effects of a disease or condition. Treatment can also refer to a method of reducing the underlying cause of the disease or condition itself rather than just the symptoms. The treatment can be any reduction from native levels and can be, but is not limited to, the complete ablation of the disease, condition, or the symptoms of the disease or condition.

The term “prevent,” “prevention” or “preventing” means inhibiting or averting symptoms associated with the target disease.

The term “therapeutically effective amount” refers to that amount of a compound, material, or composition comprising a compound of the present disclosure which is effective for producing a desired therapeutic effect, at a reasonable benefit/risk ratio applicable to any medical treatment.

The term “neurodegenerative” is generally used to describe the progressive loss of structure and/or function of neurons. Various neurodegenerative diseases, including Alzheimer's disease, Parkinson's disease, Huntington's disease, etc., occur due to neurodegenerative processes and are currently incurable, leading to the progressive degeneration and/or death of neurons. A common feature connecting multiple neurodegenerative diseases is that they all involve the accumulation of amyloid proteins, which are aggregates of fibrous proteins that share specific structural characteristics. Amyloid proteins are insoluble and arise from at least 18 types of improperly folded proteins and peptides that naturally exist in the body. These misfolded structures alter their proper conformation, causing them to incorrectly interact with each other or other cellular components, and form insoluble fibrils. To date, amyloid proteins have been associated with the pathology of over 20 serious human diseases, as the abnormal accumulation of amyloid fibrils in organs may lead to amyloidosis.

The term “inhibition of acetylcholinesterase (AChE) activity” or “acetylcholinesterase (AChE) activity inhibition” refers to the alteration of the function of acetylcholinesterase (AChE).

Red ball ginger (Zingiber zerumbet), also known as ball ginger, is a perennial herbaceous plant in the ginger family (Zingiberaceae). It is native to India and the Malay Peninsula, and its main distribution areas are currently in Southeast Asia, including India, Malaysia, Ceylon, Vietnam, Indonesia, Java, as well as Taiwan and southern China. Red ball ginger can be used for ornamental purposes in gardens or as a traditional medicinal plant, and ginger family plants have been widely used in daily diets and medicinal applications.

The main edible part of red ball ginger is the rhizome, which primarily contains triterpenoids (zerumbone, α-humulene, caryophyllene oxide) and flavonoids (kaempferol, 3-O-methyl kaempferol). It exhibits various active effects, including anti-inflammatory, anti-allergic, anti-tumor, analgesic, and antimicrobial properties. Zerumbone is the most abundant active indicator component, and is known for its therapeutic effects on metabolic syndrome, cardiovascular diseases, as well as its antioxidant, anticancer, antibacterial, anti-ulcer, anti-allergic, and immune-regulating properties. However, current commercially available products may not be able sufficiently effective due to the low content of zerumbone. Existing techniques may not achieve sufficiently efficient extraction of the triterpenoids and flavonoids from Zingiber zerumbet. The reason may lie mainly in that zerumbone being a natural terpene with low solubility in water, making it difficult to dissolve in aqueous solutions when extracted with boiled water, the most conventionally used method. As a result, products that contain water extracts have a low amount of detected zerumbone. Accordingly, the present disclosure develop different strategies of processing Zingiber zerumbet and applying the processed Zingiber zerumbet to improve or treat the above-mentioned symptoms or diseases.

The inventors established relations between the (primary) indicator components, amounts and/or ratios thereof and the efficacy of the extract or extract composite to ensure optimization of processing procedures and/or raw materials of red ball gingers.

In one embodiment, the Zingiber zerumbet rhizome extract or extract composite comprises at least 70 mg/g, at least 80 mg/g, at least 100 mg/g, at least 110 mg/g, at least 120 mg/g or at least 200 mg/g zerumbone/extract.

In one embodiment, the Zingiber zerumbet rhizome extract comprises at least 500 mg/g, at least 550 mg/g or at least 600 mg/g zerumbone/extract. In one embodiment, the Zingiber zerumbet rhizome extract comprises 500 mg/g to 1,000 mg/g, 550 mg/g to 900 mg/g, 600 mg/g to 800 mg/g, 650 mg/g to 750 mg/g, 550 mg/g to 700 mg/g zerumbone/extract, or any reasonable numeric ranges constituted by the numeric value noted herein, e.g., 500 mg/g to 800 mg/g, 650 mg/g to 800 mg/g zerumbone/extract, etc.

In any preceding aspects or embodiments, the Zingiber zerumbet rhizome extract or extract composite comprises at least 5.5 mg/g, at least 6.0 mg/g, at least 6.5 mg/g, at least 7.0 mg/g, at least 7.5 mg/g or at least 8.0 mg/g 3-O-methyl kaempferol/extract. In any preceding aspects or embodiments, the Zingiber zerumbet rhizome extract or extract composite comprises 5.5 mg/g to 15 mg/g, 6.0 mg/g to 13.5 mg/g, 6.5 mg/g to 12 mg/g, 7.0 mg/g to 11 mg/g, 7.5 mg/g to 10 mg/g or 5.5 mg/g to 9 mg/g 3-O-methyl kaempferol/extract, or any reasonable numeric ranges constituted by the numeric value noted herein, e.g., 9 mg/g to 13.5 mg/g, 5.5 mg/g to 8.0 mg/g 3-O-methyl kaempferol/extract, etc.

In any preceding aspects or embodiments, in the Zingiber zerumbet rhizome extract or extract composite, the ratio of 3-O-methyl kaempferol to zerumbone is at least 0.05, preferably at least 0.06, more preferably at least 0.07 . . . . In any preceding aspects or embodiments, in the Zingiber zerumbet rhizome extract or extract composite, the ratio of 3-O-methyl kaempferol to zerumbone is 0.05 to 0.15, 0.06 to 0.13, 0.07 to 0.12, 0.08 to 0.11 or 0.09 to 0.10, or any reasonable numeric ranges constituted by the numeric value noted herein, e.g., 0.08 to 0.15, 0.05 to 0.07, etc.

In any preceding aspects or embodiments, the Zingiber zerumbet rhizome extract is obtained by extraction with supercritical fluid carbon dioxide (CO₂).

In any preceding aspects or embodiments, the Zingiber zerumbet rhizome extract is obtained by extraction with (water) steam distillation; preferably the extract is present in an oil phase or in the form of an oily substance.

In any preceding aspects or embodiments, the Zingiber zerumbet rhizome extract is obtained by extraction of residue of the supercritical fluid CO₂ extract with an ethanolic solution.

In any preceding aspects or embodiments, the Zingiber zerumbet rhizome extract is obtained by extraction of residue of the (water) steam distillation extract with an ethanolic solution.

In any preceding aspects or embodiments, the ethanolic solution for extracting the residue is 75% ethanolic solution.

In any preceding aspects or embodiments, the Zingiber zerumbet rhizome extract or extract composite further comprises at least one component selected from the group consisting of zerumbone epoxide and kaempferol-3-O-(3,4-di-O-acetyl-α-L-rhamnopyranoside). In any preceding aspects or embodiments, the Zingiber zerumbet rhizome extract or extract composite further comprises at least one component selected from the group consisting of humulene diepoxide A, zerumbone epoxide and β-caryophyllene oxide. In any preceding aspects or embodiments, the Zingiber zerumbet rhizome extract or extract composite further comprises at least one component selected from the group consisting of zerumbone epoxide, kaempferol-3-O-(3,4-di-O-acetyl-α-L-rhamnopyranoside), humulene diepoxide A, zerumbone epoxide and β-caryophyllene oxide.

In any preceding aspects or embodiments, the Zingiber zerumbet rhizome extract or extract composite comprises (b) about 30.0 mg/g+/−10 mg/g α-humulene/extract, (c) about 35.0 mg/g+/−10 mg/g caryophyllene oxide/extract, and (d) about 10.0 mg/g+/−5 mg/g β-eudesmol/extract. In any preceding aspects or embodiments, the Zingiber zerumbet rhizome extract or extract composite comprises at least one of the following: (b) about 30.0 mg/g+/−7.5 mg/g α-humulene/extract, (c) about 35.0 mg/g+/−7.5 mg/g caryophyllene oxide/extract, (d) about 10.0 mg/g+/−3 mg/g β-eudesmol/extract, (b) about 30.0 mg/g+/−5 mg/g α-humulene/extract, (c) about 35.0 mg/g+/−5 mg/g caryophyllene oxide/extract, (d) about 10.0 mg/g+/−2 mg/g β-eudesmol/extract.

In any preceding aspects or embodiments, the Zingiber zerumbet rhizome extract or extract composite comprises (b) about 60.0 mg/g+/−10 mg/g α-humulene/extract, (c) about 55.0 mg/g+/−10 mg/g caryophyllene oxide/extract, (d) about 15.0 mg/g+/−5 mg/g β-eudesmol/extract, (e) about 40.0 mg/g+/−10 mg/g camphene/extract, and (f) about 10.0 mg/g+/−5 mg/g eucalyptol/extract. In any preceding aspects or embodiments, the Zingiber zerumbet rhizome extract or extract composite comprises at least one of the following: (b) about 60.0 mg/g+/−7.5 mg/g α-humulene/extract, (c) about 55.0 mg/g+/−7.5 mg/g caryophyllene oxide/extract, (d) about 15.0 mg/g+/−3 mg/g β-eudesmol/extract, (e) about 40.0 mg/g+/−7.5 mg/g camphene/extract, (f) about 10.0 mg/g+/−3 mg/g eucalyptol/extract, (b) about 60.0 mg/g+/−5 mg/g α-humulene/extract, (c) about 55.0 mg/g+/−5 mg/g caryophyllene oxide/extract, (d) about 15.0 mg/g+/−2 mg/g β-eudesmol/extract, (e) about 40.0 mg/g+/−5 mg/g camphene/extract, (f) about 10.0 mg/g+/−2 mg/g eucalyptol/extract.

In any preceding aspects or embodiments, in the Zingiber zerumbet rhizome extract or extract composite, the weight ratio of α-humulene to zerumbone is 0.062+/−10%, or 0.062+/−5%, or 0.062+/−3%; the weight ratio of β-eudesmol to zerumbone is 0.016+/−10%, or 0.016+/−5%, or 0.016+/−3%; the weight ratio of caryophyllene oxide to zerumbone is 0.073+/−10%, or 0.073+/−5%, or 0.073+/−3%; the weight ratio of α-humulene to zerumbone is 0.126+/−10%, or 0.126+/−5%, or 0.126+/−3%; the weight ratio of β-eudesmol to zerumbone is 0.025+/−10%, or 0.025+/5%, or 0.025+/−3%; the weight ratio of caryophyllene oxide to zerumbone is 0.107+/−10%, or 0.107+/−5%, or 0.107+/−3%; the weight ratio of camphene to zerumbone is 0.078+/−10%, or 0.078+/−5%, or 0.078+/−3%; and/or the weight ratio of eucalyptol to zerumbone is 0.027+/−10%, or 0.027+/−5%, or 0.027+/−3%.

Since zerumbone (a sesquiterpene) is considered as the primary active ingredient in Zingiber zerumbet, the extraction efficiency of zerumbone would be important. Beyond this, the inventors surprisingly found that other components in Zingiber zerumbet, such as other sesquiterpenes, flavone aglycones (e.g., 3-O-methyl kaempferol) and/or flavone glycosides (e.g., kaempferol-3-O-(3,4-di-O-acetyl-α-L-rhamnopyranoside)), may provide additive and even synergistic effects. The inventors also surprisingly found that a two-stage extraction may provide desired constitution of the active ingredients. In a specific embodiment, using a supercritical fluid (e.g., carbon dioxide, CO₂) or (water) steam distillation to treat Zingiber zerumbet can obtain extracts comprising active ingredients, primarily zerumbone; the residue after said supercritical fluid extraction or steam distillation can be further extracted with an ethanolic solution to obtain residual or other active ingredients therefrom, and the extracts can be combined to provide an extract composite.

In any preceding aspects or embodiments of the process of making Zingiber zerumbet rhizome extract composite, the weight ratio recited in step (c) is about 1:1.2, about 1:1.5, about 1:1.2, about 1:3, about 1:4, about 1:5, about 1:8, about 1:9 or about 1:9.5, or any reasonable numeric ranges constituted by the values noted herein, e.g., from 1:9 to 1:1.5, 1:3 to 1:1.2, 1:9.5 to 1:4, etc.

The extracts can be analyzed by GC-MS and/or HPLC for qualitative and quantitative analysis. Without being bound to theory, after establishment of the growth process of red ball ginger and changes in rhizome indicator components, harvesting periods of red ball gingers can be optimized. The processes of extraction of rhizome of red ball ginger can also be designed to obtain extracts comprising suitable or desired (active) components in suitable or desired amounts. In addition, extracts obtained by processing different portions of red ball gingers and/or red ball gingers harvested in different periods can also thus be combined to provide extract composites having suitable or desired (active) components in suitable or desired amounts, as described herein.

In particular, the inventors conducted qualitative and quantitative analysis of extracts of Zingiber zerumbet rhizome obtained by using 95% ethanolic solution, followed by ethyl acetate extraction as reference. The extract was then subjected to HPLC chromatography under gradient elution with hexane/ethyl acetate (1:0 to 0:1), followed by ethyl acetate/methanol (1:0 to 0:1) to give various fractions. The fractions were subjected to further separation of components with different columns (silica, resin, reversed-phased column, etc.) for NMR identification. Nine components were identified: α-humulene, zerumbone, zerumbone epoxide, 3,4′-di-O-methyl kaempferol, 3-O-methyl-kaempferol, kaempferol-3-O-(2,4-di-O-acetyl-α-L-rhamnopyranoside), kaempferol-3-O-(3,4-di-O-acetyl-α-L-rhamnopyranoside), kaempferol-3-O-α-L-rhamnopyranoside and kaempferol-3-O-(4-O-acetyl-α-L-rhamnopyranoside).

It is clear that prevention of a disease or disorder is better than treatment thereof. In addition to basic nutrition, the public is increasingly focusing on health care in order to prevent potential risk of suffering diseases or disorders. Applicant finds that effective proprietary ingredients that provide specific extracts or combinations to be used as raw materials for food and health products can be developed by cultivating Zingiber zerumbet, in particular those harvested during specific periods in Taiwan. Furthermore, the applicant finds that the values of these processed agricultural products can be enhanced as they have potential to be used or processed as pharmaceutical-grade products, offering joint health support and neuroprotection to prevent or treat osteoarthritis and neurodegenerative diseases associated with cognitive decline.

As zerumbone in the Zingiber zerumbet may exhibit anti-inflammatory and anti-oxidation effects, Zingiber zerumbet rhizome extract as described herein has potential applications in various related healthcare and medical objectives, including but not limited to improvement of cognitive dysfunctions (in particular in neurodegenerative diseases) in a subject, prevention and/or treatment of osteoarthritis in a subject, etc.

In various aspects or embodiments, the cognitive dysfunctions comprises age-associated memory impairment, age-associated cognitive decline or mild cognitive Impairment.

In any preceding aspects or embodiments, the neurodegenerative diseases is Parkinson's disease (PD), Alzheimer's disease (AD), Huntington's disease (HD), dementia, prior diseases, motor neuron disease, spinocerebellar Ataxia (SCA), amyotrophic lateral sclerosis (ALS) or Lewy body diseases.

In any preceding aspects or embodiments, the osteoarthritis is primary osteoarthritis, occupational osteoarthritis and/or secondary osteoarthritis.

In any preceding aspects or embodiments, the Zingiber zerumbet rhizome extract or extract composite exhibits an inhibitory effect of acetylcholinesterase (AChE) activity.

In any preceding aspects or embodiments, the Zingiber zerumbet rhizome extract or extract composite exhibits an inhibitory effect of aggregation of amyloid beta (β-amyloid, Aβ) peptide 1-42.

In any preceding aspects or embodiments, the cognitive dysfunctions comprises age-associated memory impairment, age-associated cognitive decline or mild cognitive Impairment.

In any preceding aspects or embodiments, the neurodegenerative diseases is Parkinson's disease (PD), Alzheimer's disease (AD), Huntington's disease (HD), dementia, prior diseases, motor neuron disease, spinocerebellar Ataxia (SCA), amyotrophic lateral sclerosis (ALS) or Lewy body diseases.

In any preceding aspects or embodiments, the amount of at least one oxidative stress indicator is reduced after the administration of the Zingiber zerumbet rhizome extract or extract composite.

In any preceding aspects or embodiments, in the method of improving cognitive dysfunctions in neurodegenerative diseases in a subject, the amount of at least one oxidative stress indicator is reduced after the administration. In various embodiments, the oxidative stress indicators can be selected from the group consisting of malondialdehyde (MDA), total glutathione contents, glutathione peroxidase activity, glutathione reductase activity, or advanced glycation end-products (AGEs), particularly malondialdehyde (MDA).

In any preceding aspects or embodiments, the Zingiber zerumbet rhizome extract or extract composite promotes growth of synapse and/or exhibits neuroprotection.

In any preceding aspects or embodiments, the Zingiber zerumbet rhizome extract or extract composite exhibits an anti-inflammatory effect.

In any preceding aspects or embodiments, the Zingiber zerumbet rhizome extract or extract composite exhibits an inhibitory effect of non-enzymatic glycation of proteins.

The herbal compositions and the extracts described herein are ideally administered orally and can be formulated into foods using methods commonly recognized in the field. These herbal compositions and extracts can also be utilized as dietary supplements or medicinal preparations, available in solid, semi-solid, or liquid forms. They may contain the herbal compositions and the extracts described herein, either on their own or mixed with suitable organic or inorganic carriers or excipients for external, enteral, or parenteral use. The herbal compositions and the extracts described herein can be combined with standard non-toxic, pharmaceutically acceptable carriers for various forms, including tablets, pellets, capsules, suppositories, solutions, emulsions, suspensions, and other appropriate formats. The formulations of this invention may include carriers such as talc, water, glucose, lactose, gum acacia, gelatin, mannitol, starch paste, magnesium trisilicate, corn starch, keratin, colloidal silica, potato starch, urea, and other suitable carriers for creating solid, semi-solid, or liquid preparations. Additionally, auxiliary agents such as stabilizers, thickeners, coloring agents, and fragrances may be incorporated.

To create solid formulations like tablets or capsules, the herbal compositions and the extracts described herein are blended with a pharmaceutical carrier (such as traditional tableting components like corn starch, lactose, sucrose, sorbitol, talc, stearic acid, magnesium stearate, dicalcium phosphate, or gums) along with other pharmaceutical diluents. The solid preformulation composition may be then divided into unit dosage forms recognized in the field, preferably in the form of capsules.

Liquid formulations can be administered orally. These include aqueous solutions, flavored syrups, aqueous or oil-based suspensions, and flavored emulsions using edible oils such as cottonseed, sesame, coconut, or peanut oil, as well as elixirs and other pharmaceutical vehicles. For aqueous suspensions, suitable dispersing or suspending agents may include synthetic natural gums like tragacanth, acacia, alginate, dextran, sodium carboxymethyl cellulose, methylcellulose, polyvinylpyrrolidone, or gelatin. Liquid preparations intended for oral administration can be provided as dry products that require reconstitution with water or other appropriate vehicles prior to use. These preparations can be created using standard methods and may include pharmaceutically acceptable additives such as suspending agents (e.g., sorbitol syrup, methyl cellulose, or hydrogenated edible fats), emulsifying agents (e.g., lecithin or acacia), non-aqueous vehicles (e.g., almond oil, oily esters, or ethyl alcohol), preservatives (e.g., methyl or propyl p-hydroxybenzoates or sorbic acid), and artificial or natural colors and/or sweeteners.

### EXAMPLES

The following examples are provided to make the present invention more comprehensible to those of ordinary skill in the art to which the present invention pertains, but are not intended to limit the scope of the invention.

### Example 1—Preparation and Analysis of Red Ball Ginger (Zingiber zerumbet) Extracts and Extract Composites

### Steam Distillation Method

About 99.5 kilograms of commercially available dried Zingiber zerumbet (water content of about 5%) and about 87.5 kilograms of fresh Zingiber zerumbet (water content of about 60%) were subjected to steam distillation for 16 hours (the temperature was raised in the first four hours, kept stable in the subsequent six hours and lowered under a negative pressure for six hours). Essential oils were collected from the water vapor during the distillation process. The Zingiber zerumbet remained soaked in the water and was subjected to steam distillation next day for 14 hours to collect essential oils. The essential oils were combined as the extraction product (J).

The residue obtained from steam distillation was further extracted with 10× volume of 75% ethanolic solution under reflux at 65° C. to 75° C. for two hours, and the process was repeated again. The filtrates were combined, concentrated and lyophilized to give the extraction product (K).

### Supercritical Fluid Carbon Dioxide (CO₂) Extraction

About 99.5 kilograms of commercially available dried Zingiber zerumbet (water content of about 5%) was ground and sieved with 60 #sieving screens (60 mesh, about 250 μm) to provide powdered Zingiber zerumbet. The powdered Zingiber zerumbet was subjected to a supercritical fluid device for extraction by CO₂ under 50° C. at a pressure of 4650 psi to give the extraction product (I).

The residue obtained from supercritical fluid CO₂ extraction was further extracted with 10× volume of 75% ethanolic solution under reflux at 65° C. to 75° C. for two hours, and the process was repeated again. The filtrates were combined, concentrated and lyophilized to give the extraction product (M).

### Extract Composites

Desired ratios (e.g., 1:9 or 2:8) of the product (I) or (J) and the lyophilized product (M) or (K) were dissolved together in methanol. After being thoroughly mixed, the solution was concentrated and lyophilized to give the extract composite (I/M or J/K).

### GC-MS Analysis

Helium gas was used as carrier gas, and electron ionization (EI) (70 eV) was adopted for ionization; the scanned range of m/z was from 29 to 500 and peak areas (with generalization) were used for quantitation of relative percentage of analytes. The mass data was compared with NIST 14 and Wiley 275 databases using Mass Hunter for identification of the analytes.

### HPLC Qualitative and Quantitative Analysis

A 1 mg sample was diluted with 1 mL HPLC-grade methanol and then subjected to ultrasonication for 30 minutes to completely dissolve the sample. The solution was filtered with 0.45 μm filter membrane and the filtrate was subjected to HPLC analysis. A reversed phase column (RP-18e) was used and the mobile phase was 0.05% TFA-CH₃CN (0-10 min, 65:35; 20-30 min, 35:65; 35-40 min, 0:100; 41 min, 65:35).

The content of zerumbone in commercially available products of Zingiber zerumbet ((a) prepared drug in pieces or decoction pieces, (b) Zingiber zerumbet powders, (c) food made of ethanolic extract of Zingiber zerumbet and (d) fermentation liquid of Zingiber zerumbet) was also analyzed.

### Results

The results of GC-MS analysis of the components in the extracts are shown in Table 1:

TABLE 1

| | | | | | | % Area | | | | |
|---|---|---|---|---|---|---|---|---|---|---|
| No. | Compound | Chemical Class | RI_{L} | RI | RT | J | I | J(2) | I(2) | Identification |
| 1 | α-Thujene | Monoterpene | 931 | 960 | 12, 18 | Tr | — | tr | — | MS, RI |
| 2 | α-Pinene | Monoterpene | 939 | 971 | 12, 74 | 0.53 | — | tr | — | MS, RI |
| 3 | Camphene | Monoterpene | 953 | 988 | 13, 57 | 2.6 | — | 1.4 | — | MS, RI |
| 4 | δ-3-carene | Monoterpene | 1011 | 1027 | 16, 61 | 0.16 | — | tr | — | MS, RI |
| 5 | Myrcene | Monoterpene | 991 | 1064 | 17, 49 | tr | — | tr | — | MS, RI |
| 6 | Limonene | Monoterpene | 1031 | 1068 | 17, 71 | 0.17 | — | tr | — | MS, RI |
| 7 | Eucalyptol | Monoterpene | 1039 | 1071 | 17, 84 | 1.08 | — | 0.5 | — | MS, RI |
| 8 | α-Terpinolene | Monoterpene | 1088 | 1131 | 21, 38 | 0.26 | — | 0.14 | — | MS, RI |
| 9 | Camphor | Monoterpene | 1143 | 1188 | 23, 67 | 1.74 | 0.19 | 0.43 | 0.22 | MS, RI |
| 10 | Camphene | Monoterpene | 1148 | 1196 | 24, 08 | tr | — | — | — | MS, RI |
| | hydrate | | | | | | | | | |
| 11 | β-Caryophyllene | Sesquiterpene | 1420 | 1468 | 36, 16 | 0.48 | 1.21 | 0.41 | 0.4 | MS, RI |
| 12 | α-Humulene | Sesquiterpene | 1449 | 1505 | 37, 67 | 7.94 | 9.5 | 6.12 | 7.91 | MS, RI |
| 13 | trans- | Sesquiterpene | 1594 | 1615 | 41, 91 | tr | — | — | — | MS, RI |
| | Sesquilavandulyl | | | | | | | | | |
| | acetate | | | | | | | | | |
| 14 | Caryophyllene | Sesquiterpene | 1581 | 1637 | 42, 70 | 1.64 | 0.19 | 0.59 | 0.57 | MS, RI |
| | oxide | | | | | | | | | |
| 15 | Humulene oxide I | Sesquiterpene | 1602 | 1654 | 43, 36 | 4.06 | 1.7 | 4.21 | 1.66 | MS, RI |
| 16 | Humulene oxide II | Sesquiterpene | 1602 | 1654 | 43, 78 | 4.11 | 1.59 | 4.03 | 1.89 | MS, RI |
| 17 | β-Eudesmol | Sesquiterpene | 1650 | 1678 | 45, 46 | 0.4 | 0.15 | 0.4 | 0.4 | MS, RI, |
| | | | | | | | | | | HPLC |
| 18 | (E,E)-Farnesol | Sesquiterpene | 1686 | 1624 | 46, 39 | 0.39 | — | — | — | MS, RI |
| 19 | Palustrol | Diterpene | 1566 | 1677 | 47, 92 | tr | tr | — | — | MS, RI |
| 20 | Zerumbone | Sesquiterpene | 1733 | 1728 | 48, 30 | 58.79 | 62.97 | 64.3 | 64.41 | MS, RI, |
| | | | | | | | | | | HPLC |

| | | | | |
|---|---|---|---|---|
| Total | 84.56 | 77.59 | 82.53 | 77.46 |

RI = Experimental Retention Indices obtained relative to C8-C20 Alkane series on the column
RI_{L} = Retention Indices found and reported in literature (Adam, 2007) for equivalent column
UV = High Pressure Liquid Chromatography UV Visible, Tr = Trace (<0.1%)
J and I: commercially available zingiber zerumbet, slices (water content of about 60%) or decoction pieces (water content of about 5%); J(2) and I(2): freshly harvested zingiber zerumbet, slices (water content of about 60%) or decoction pieces (water content of about 5%).

The quantitative analysis result of HPLC analysis of main components in the extracts are summarized in Table 2:

TABLE 2

| No. | Component | Extract I | | Extract J |
|---|---|---|---|---|
| 1a | Camphene | ND | | 40.8 mg/g |
| 2a | Eucalyptol | ND | | 13.87 mg/g |
| 5a | α-Humulene | 33.65 | mg/g | 65.61 mg/g |
| 6a | Caryophyllene oxide | 39.44 | mg/g | 55.94 mg/g |
| 9a | β-Eudesmol | 8.84 | mg/g | 12.95 mg/g |
| 10a | Zerumbone | 543.068 | mg/g | 522.3 mg/g |
| 15a | Total polyphenol | ~8.7 | mg gallic acid/g | |
| 16a | Total flavonoid | ~12 | mg rutin/g | |

HPLC-UV analysis of commercially available products showed the following zerumbone concentrations: (a) prepared drug in pieces or decoction pieces: 15 to 56 mg/g; (b) Zingiber zerumbet powders: about 31.0 mg/g; (c) food made of ethanolic extract of Zingiber zerumbet: about 4.5 mg/g; and (d) fermentation liquid of Zingiber zerumbet: undetected; the concentration is far below that presented in Extracts I and J.

Analysis of extract composites reveal that zerumbone and 3-O-methyl kaempferol are also present in sufficient amounts (see Table 3):

TABLE 3

| Sample No. | Zerumbone (mg/g) | 3-O-methyl kaempferol (mg/g) |
|---|---|---|
| Extract I | 667.13 | ND |
| Extract J | 611.16 | ND |
| Extract K | 47.50 | 2.39 |
| Extract M | 3.38 | 3.89 |
| Extracts (I + M = 1:9) | 126.89 | 9.975 |
| Extracts (I + M = 2:8) | 122.87 | 8.441 |
| RAW | 31.373 | 0.579 |

RAW: zingiber zerumbet powders (sieved by 60# sieving screen)

The results show that the claimed extracts and extract composites contain significantly high content of zerumbone/3-O-methyl kaempferol over raw Zingiber zerumbet powders and 95% ethanolic extracts. In addition, various components are present in sufficiently high amounts to be adopted as indicators for quality control.

### Example 2—Evaluation of Inhibition of Acetylcholinesterase (AChE) Activity

Commercially available acetylcholinesterase (AChE) was used for hydrolysis. Thiocholine generated by the hydrolysis of acetylthiocholine iodide can react with DTNB (5,5′-dithiobis-(2-nitrobenzoic acid)) and absorbance at a wavelength of 405 nm (A405 nm) was measured when extracts were added. If the amount of thiocholine produced decreases, the yellow product formed with DTNB will also decrease, resulting in a decrease in A405 nm relative to the control group. This allows for the determination of the concentration required for 50% inhibition (IC₅₀) of each screened target.

In a 96-well plate, the 9 μL containing 0.025 μg (500 nmol/min/μg) of AChE was added along with 1 μL of different concentrations of the extract, and 50 μL of 100 mM (pH 7.5) phosphate buffer. After thorough mixing, the reaction was conducted at 4° C. for 15 minutes. Then, 20 μL of 1 mM acetylthiocholine iodide is added, and the reaction was allowed to proceed at room temperature for 15 minutes. Following this, 20 μL of 2 mM DTNB was added, and the reaction was again allowed to proceed at room temperature for 15 minutes. The absorbance at 405 nm was measured using an ELISA reader. The formula for AChE inhibition is as follows: [(blank group−blank)−(sample group−sample blank)]/average value of the blank group*100%.

The inhibitory effect of acetylcholinesterase (AChE) activity of the tested samples is expressed by IC₅₀ and shown in Table 4 below. Incidentally, the IC₅₀ of zerumbone under the same assay was 384 μM.

TABLE 4

| Sample No. | IC₅₀ (μg/mL) |
|---|---|
| Extract I | 186.64 |
| Extract J | 957.69 |
| Extract K | 3941.65 |
| Extract M | 1797.43 |

### Example 3—Evaluation of Inhibition of Aggregation of β-Amyloid Peptide 1-42

Commercially available amyloid beta peptide (1-42) (Aβ) was dissolved in DMSO to prepare a stock solution thereof (5 mM). Aβ was diluted to 10 μM with PBS while keeping the concentration of amyloid beta peptide constant, and proportionally increasing different concentrations of extracts. The samples were placed in a black 96-well plate and incubated at 37° C. under shaking for 24 hours. Portions of 80 μL of the reaction solution were taken for addition of 10 μL of 100 mM thioflavin T (ThT) and Tris-glycine buffer (pH 8.5). Fluorescence absorption (Ex440 nm/Em486 nm) was then measured. The value measured with ThT for Aβ (10 μM) aggregated for 24 hours was set 100 as the blank test, which was used to evaluate the ability of the tested drugs to inhibit amyloid beta peptide aggregation.

The inhibitory effect of aggregation of β-amyloid peptide 1-42 of the tested samples is expressed by IC₅₀ and shown in Table 5 below. Incidentally, the IC₅₀ of zerumbone under the same assay was 78.9 μM.

TABLE 5

| Sample No. | IC₅₀ (μg/mL) |
|---|---|
| Extract I | 71.75 |
| Extract J | >200 |
| Extracts (I + M = 1:9) | 15.86 |
| Extracts (I + M = 2:8) | 17.63 |

Though Extract M comprises less zerumbone than Extract I, a combination of Extract I and Extract M provides better effects of inhibition of aggregation of β-amyloid peptide 1-42 than Extract I alone. Without being bound to theory, additional active ingredients (such as 3-O-methyl kaempferol) in the Zingiber zerumbet extracts in combination with zerumbone may provide additive, even synergistic effects.

### Example 4—Evaluation of Enhancement/Promotion of Outgrowth of PC12 Cells

A poly-L-lysine (PLL) solution was prepared of which 50 μL was added to each well of a 96-well plate. After it was allowed to stand in the incubator for 1 hour, the excess PLL was removed and washed three times with sterilized ddH₂O. The plate was placed in a sterile workbench for more than 3 hours to dry. PC-12 cells were counted for preparing a cell suspension at a concentration of 5×10⁴ cells/mL using RPMI1640 medium (containing 10% HS and 5% FBS), and 100 μL of the cell suspension was inoculated into each well of the PLL-treated 96-well plate, achieving a density of 5×10³ cells/well. The samples were incubated in the incubator for 24 hours to allow the cells to adhere. After images were captured using an inverted optical microscope imaging system, the cells were divided into three groups: blank group, positive control group, and sample group. RPMI1640 medium (containing 0.5% FBS) with 0.1% DMSO was added to the blank group, 100 ng/mL NGF was added to the positive control group, and samples of various concentrations were added to the sample group. Images were captured on the third and sixth days using the inverted optical microscope imaging system, and fresh RPMI1640 medium (containing 0.5% FBS) with 0.1% DMSO (for blank group), 100 ng/mL NGF (for positive control group) and samples of various concentrations (for sample group) were used for replacement in each well. Alternatively, the samples were cultured with various concentrations of zerumbone (sample group) in OptI/MEM (containing 0.5% FBS) for six days, and images were captured using the inverted optical microscope imaging system.

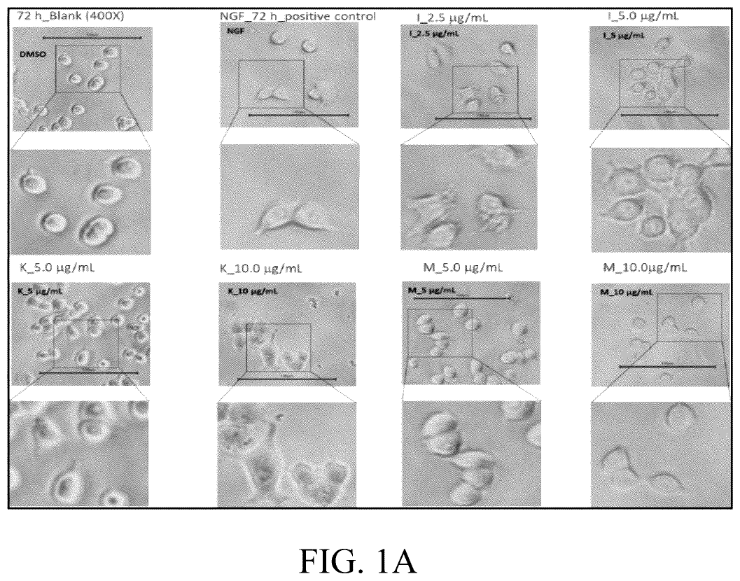

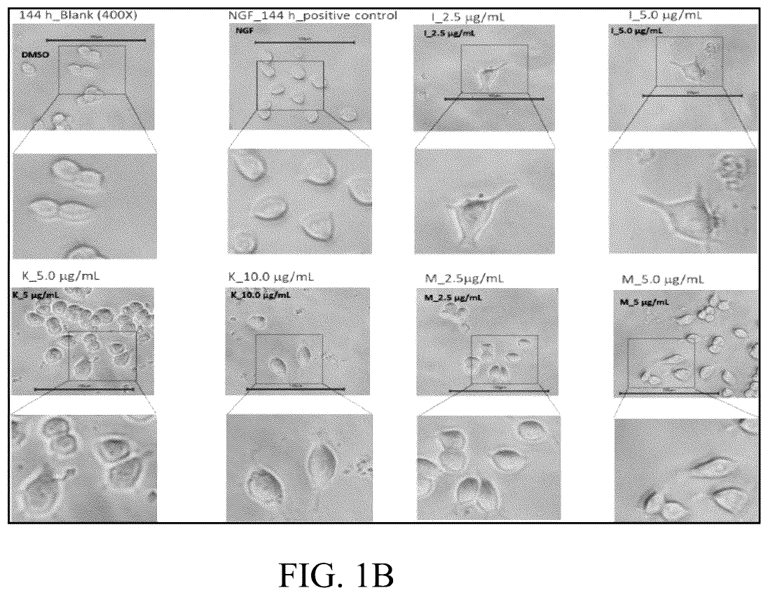

FIG. 1 shows that the captures of Extracts I, K, and M cultured for 72 hours (FIG. 1A) and 144 hours (FIG. 1B) exhibited similar neurotrophic factor activity, promoting the growth of PC12 cell neurites, as shown in the optical microscope images (400×) with local magnification (labeled box). After 72 hours (three days) or 144 hours (six days), Extract I (2.5, 5.0 g/mL), Extract K (5.0, 10.0 μg/mL) and Extract M (5.0, 10.0 μg/mL for 72-h; 2.5, 5.0 μg/mL for 144-h) exhibited obvious outgrowth of PC12 cells similar or even superior to the NGF group at 100 ng/mL.

### Example 5—Evaluation of Neuroprotection Effects

SH-SY5Y human neuroblastoma cells were used as a neuronal cell model, hydrogen peroxide was utilized to induce cell death and synaptic damage, and the extracts were evaluated for their ability to improve cell viability and reduce synaptic damage. A PLL treated 96-well plate was provided based on the process described in Example 5. SH-SY5Y cells were counted for preparing a cell suspension of 1×10⁵ cells/mL using DMEM/F-12 (containing 10% FBS), and 100 μL of this suspension was seeded into the PLL-treated 96-well plate, achieving a density of 1×10⁴ cells/well. The cells were then cultured in the incubator for 24 hours to allow adherence. After images were captured using an inverted optical microscope imaging system, the cells were divided into three groups: blank group, control group, and sample group. DMEM/F-12 (containing 10% FBS) with 0.1% DMSO (for the blank and control groups), samples of various concentrations (for the sample group) were added for each group, and the cells were cultured in the incubator for 24 hours. The existing culture medium was then removed, the control and sample groups were treated with culture medium containing 100 μM (Group A) or 200 μM (Group B) H₂O₂, while the blank group received DMEM/F-12 (containing 10% FBS) without H₂O₂, and the samples continued to be cultured in the incubator for an additional 24 hours. Images were recorded using the inverted optical microscope imaging system to observe changes in cell synapses. The existing culture medium was removed, and DMEM/F-12 (containing 10% FBS) with 0.5 mg/mL of 3-(4,5-Dimethylthiazol-2-yl)-2,5-diphenyltetrazolium bromide (MTT) was added as replacement. After 4 hours of incubation, the medium was removed, and 100 μL of DMSO was added to dissolve the purple crystals. The absorbance at 570 nm was measured using a microplate reader, and cell viability was calculated.

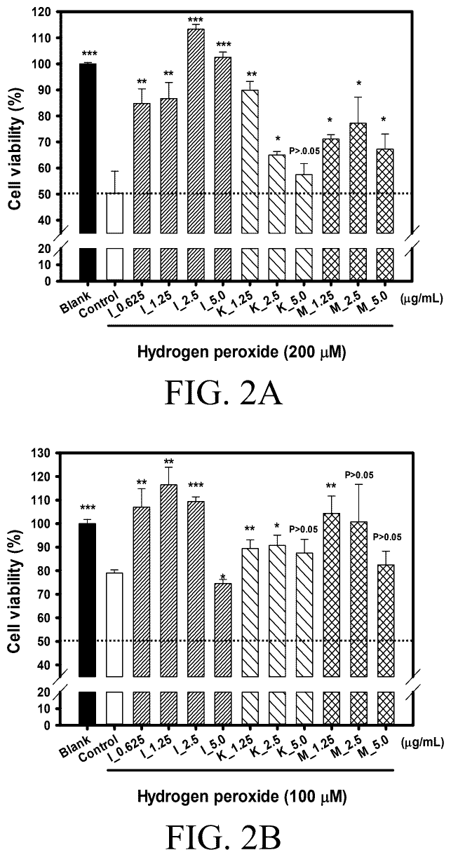

Extract I (0.625, 1.25, 2.5, 5 g/mL), Extract K (1.25, 2.5 μg/mL) and Extract M (1.25, 2.5, 5 μg/mL) were introduced after induction of cell death. The results are shown in FIGS. 2A and 2B. In Group A, the cell viability in the control group decreased to 50.35%, and all samples treated with the extracts exhibited significant increases in cell viability as compared to the control group (*** p<0.001, ** p<0.01, *p<0.05). In Group B, the cell viability in the control group decreased to 78.96%, and all samples treated with the extracts also exhibited significant increases in cell viability as compared to the control group (*** p<0.001, ** p<0.01, *p<0.05). In addition, the length of the cell synapses in the samples treated with the extracts also significantly recovered, and the cells exhibited differentiated patterns.

### Example 6—In Vitro Assessment of Inhibitory Ability on Non-Enzymatic Glycation of Proteins

Galactose undergoes non-enzymatic glycation of proteins more rapidly than glucose, so bovine serum albumin/galactose was chosen as the model for non-enzymatic protein glycation. Bovine serum albumin (2 mg/mL), 1M galactose, and samples of varying concentrations were reacted at 37° C. for 5 days. Protein staining was performed using SDS-PAGE, and immunostaining was conducted using western blotting. A primary antibody [anti-carboxymethyl-lysine (anti-CML) antibody (ab27684, Abcam)] was diluted 1000 times, and a secondary antibody conjugated to alkaline phosphatase was also diluted 1000 times. Staining was carried out using BCIP/NBT, and quantitative analysis was performed using Image J.

Using immunostaining analysis to measure the levels of CML. The control group was set at 100% CML generation, and the addition of Extracts I, K, and M (at 0.2 mg/mL) reduced the CML generation to 42.2%, 26.7%, and 25%, respectively.

### Example 7—Evaluation of Effect on Improving Cognitive Dysfunctions in Animal Model

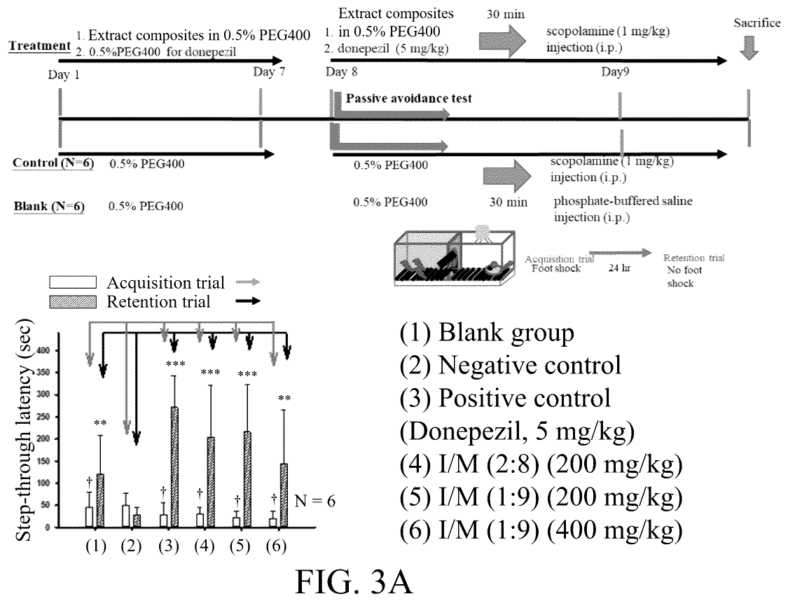

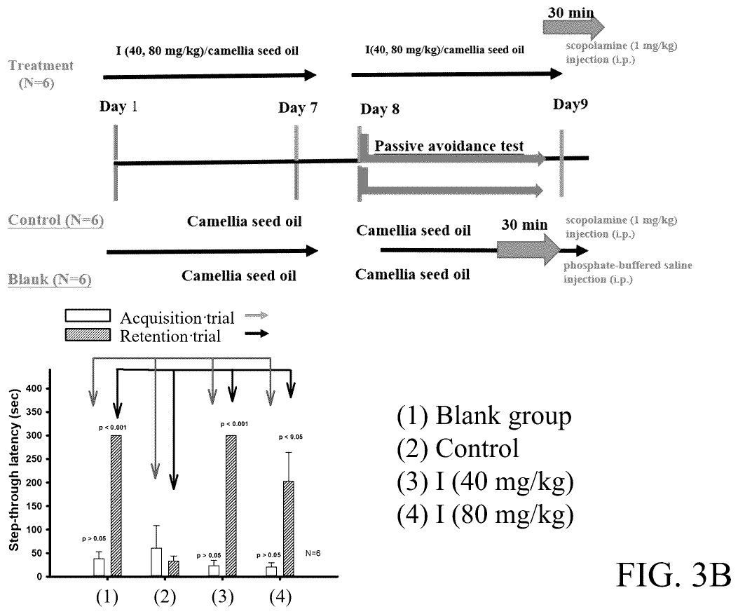

The mouse model of transient amnesia induced by scopolamine was used to evaluate the effect of the extracts on improving cognitive dysfunctions. The protocol and test results are shown in FIGS. 3A and 3B. Donepezil (positive control) and Extract I and Extract M at weight ratios of 1:9 and 2:8 (i.e., 1:4) of (I/M) (tested groups) were used in the animal model. Donepezil and the mixtures of extracts and were homogeneously suspended in 0.5% PEG400 solvent (FIG. 3A) or in camellia seed oil (FIG. 3B) before administration. In the tested groups, mice were fed with the extract composite compositions (200 or 400 mg/kg) once a day for seven days. On Day (acquisition trial) 8, mice were fed with the extract composite compositions (200 or 400 mg/kg) (tested groups) or Donepezil (5 mg/kg) (positive control group); after thirty minutes, mice in each group were then intravenously injected with scopolamine (1 mg/kg) to induce memory loss. In the blank group, phosphate-buffered saline, instead of scopolamine, was injected into the mice on Day 8. In the negative control group, only scopolamine was injected into the mice on Day 8. The protocol and test results are shown in FIG. 3A. Extract I in varying amounts (40 or 80 mg/kg) was also used in similar tests, and the protocol and test results are shown in FIG. 3B. The results reveal that on Day 8 (acquisition trial), there was no significant difference in step-through latency between the negative control group and each of the tested groups, the blank group and the positive control group. However, on Day 9 (retention trial), significant differences in step-through latency between the negative control group and each of the tested groups and the positive control group were observed (*** p<0.001, **p<0.01, *p<0.05, †p>0.05). Hence, Zingiber zerumbet rhizome extracts and extract composites were shown to improve, and even prevent, memory loss and learning impairments in mice with cognitive dysfunctions (induced by scopolamine), as the extracts or extract composites were fed to the mice before the onset of cognitive dysfunction.

A positive result was also obtained by feeding zerumbone (40 mg/kg) to the mice in the same model.

### Example 8—Evaluation of Effect on Delaying Aging, Improving Oxidative Stress and Learning Ability in Animal Model

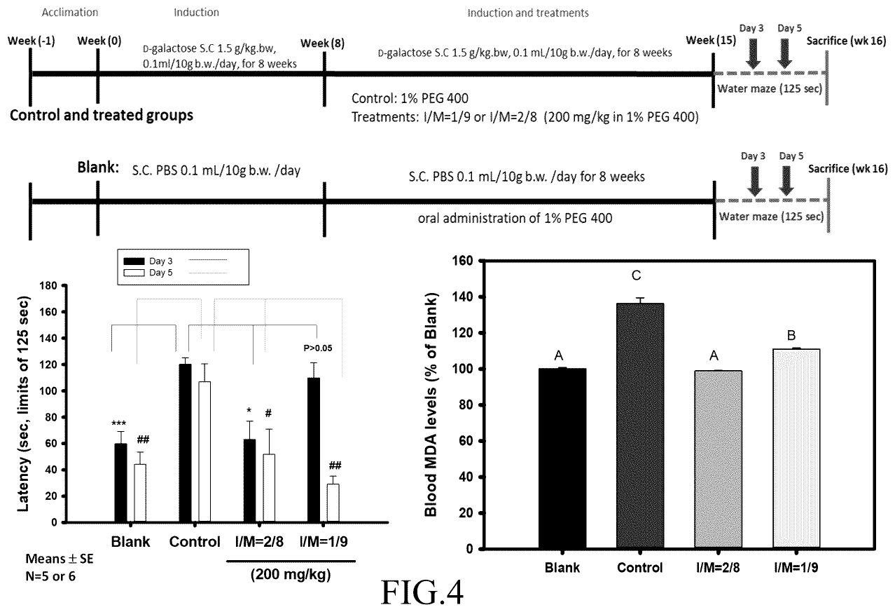

A galactose-induced oxidative stress aging mouse model was used for evaluating the improvement of oxidative stress by MDA levels and learning ability by Morris water maze. The protocol and test results are shown in FIG. 4. The effects of the compositions I/M=1/9 and 2/8 (200 mg/kg) on delaying aging were evaluated. The results showed that the blank group significantly and rapidly found the platform on both the third day (16wk-3rd) and the fifth day (16wk-5th), showing a significant difference compared to the control group. The sample group fed with I/M=2/8 also significantly and rapidly found the platform on both the third day (16wk-3rd) and the fifth day (16wk-5th), with a significant difference compared to the control group. The sample group fed with I/M=1/9 significantly and rapidly found the platform on the fifth day (16wk-5th), showing a significant difference compared to the control group. These results indicate that the extract composites I/M=1/9 and 2/8 (200 mg/kg) have the potential to improve spatial memory and learning ability in the Morris water maze, delay aging effects, and reduce the oxidative stress marker malondialdehyde (MDA) levels.

H & E staining was also used on mouse brain sections for observation. The control group showed significant degeneration and necrosis of brain cells in the hippocampal dentate gyrus, along with a moderate infiltration of gemistocytes (obese-type astrocytes) in the meninges extending into the brain parenchyma, accompanied by gliosis and perivascular cuffing. In contrast, the mice fed with galactose for eight weeks, which induced increased free radicals and oxidative stress, exhibited a significant improvement and reduction in the number of necrotic brain cells, particularly in the group fed with an I/M ratio of 2/8, where the brain cells returned to a state similar to the control group, with no significant pathological changes observed in the other groups.

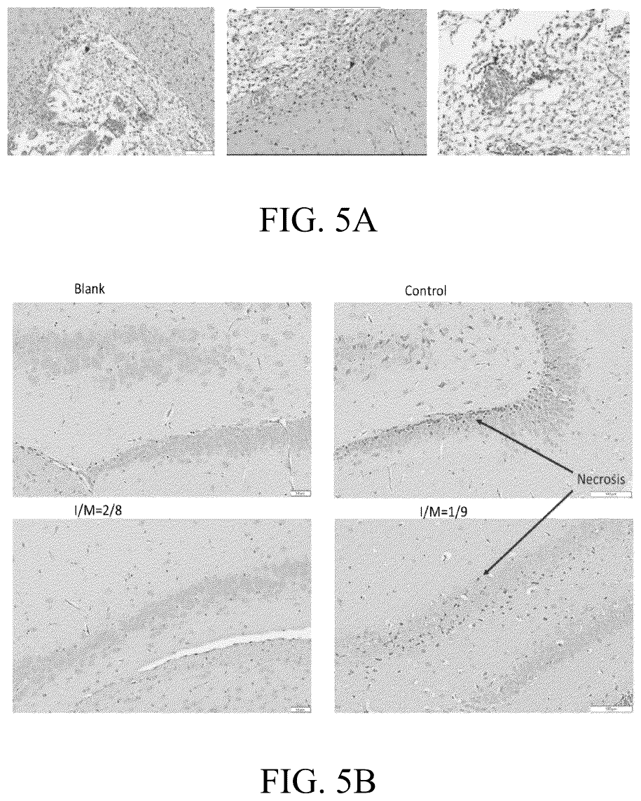

FIG. 5A shows that, in the control group of mice subjected to galactose-induced oxidative stress aging, there was a significant amount of neuronal degeneration and necrosis (the middle part of FIG. 5A) in the hippocampus, specifically in the dentate gyrus, along with a moderate infiltration of gemistocytic astrocytes (the left part of FIG. 5A) into the meninges and deeper into the brain parenchyma, accompanied by gliosis and perivascular cuffing (the right part of FIG. 5A). FIG. 5B shows the results of the blank group, the galactose-induced control group, and the intervention group I/M mixture (w/w=1/9 and 2/8). The arrows indicate areas of neuronal degeneration and necrosis in the dentate gyrus of the hippocampus in the control group.

Hence, Zingiber zerumbet rhizome extracts and extract composites were shown to delay aging, reduce oxidative stress, and enhance learning ability.

### Example 9—Evaluation of Anti-Inflammatory Effects

### DPPH Free Radical Scavenging Assay

50 μL of different concentration sample solutions were added to a 96-well plate, followed by the addition of 150 μL of 200 μM DPPH/MeOH. The mixture was thoroughly mixed and allowed to react in the dark at room temperature for 30 minutes. The absorbance at a wavelength of 515 nm was measured using a microplate reader. The calculation formula for the scavenging rate is: scavenging activity (%)=(control−sample)/control×100%.

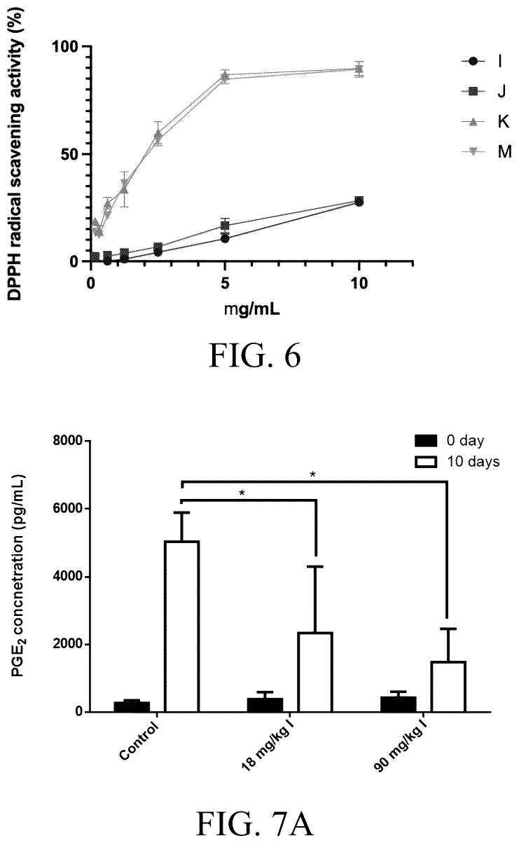

Extracts I, J, K and M were used in this assay. The IC₅₀ values for the DPPH scavenging effects of M and K are 2.20 and 2.37 mg/mL, respectively. In addition, IM1 (I:M=1:9), and IM2 (I:M=2:8) exhibit a very strong DPPH scavenging effect, with IC₅₀ values of 3.32, and 2.85 mg/mL, respectively (see, FIG. 6). This reveals the potential of using extract composites to provide better anti-oxidative effect.

### RAW 264.7 Macrophage Model

RAW 264.7 cells were seeded at a density of 4.0×10⁵/ml in a 96-well plate. After 24 hours, samples at a concentration of 200 μg/mL, including their extracts and active components, along with LPS at 100 ng/ml, were added for 18 hours. Afterward, 100 μL of the supernatant was collected and mixed with 100 μL of Griess reagent for testing. The absorbance for NO was measured at 530 nm using an ELISA reader, and an EIA kit was used to detect the PGE₂ content. Samples that inhibited the production of NO and PGE₂ were further analyzed using Western Blot to assess the expression levels of iNOS and COX-2 in the RAW 264.7 cells.

Extract I, Extract composites of I/M (1:9), and I/M (2:8) inhibited NO production, with IC₅₀ values of 8 μg/mL, 2.47 μg/mL and 3.77 μg/mL, respectively. The inhibitory effect of Extract composites were superior to the use of Extract I alone in the inhibitions of NO productions. Additionally, Extracts I and J inhibited LPS-induced PGE₂ production in RAW 264.7 cells, with IC₅₀ values of 6.64 μg/mL and 4.48 μg/mL, respectively. Extracts I and J were able to inhibit iNOS at a concentration of 3 μg/mL or higher, and they inhibited COX-2 protein expression at a concentration of at least 12 μg/mL.

### SW 1353 Chondrocyte Model

SW 1353 cells were seeded at a density of 2.0×10⁵/mL in 6-well plates. After 24 hours, the Extracts and IL-1β (at 10 ng/ml) were added (for 24 hours). Real-time polymerase chain reaction (RT-PCR) experiments were conducted to analyze the expression levels of IL-6 and MMP-13. Immunofluorescence staining was used to observe the expression of NF-κB and MMP-3.

The results showed that Extracts I and J (1.25, 2.5, and 5 μg/mL) significantly inhibited the mRNA expression of MMP-13 and IL-6 under non-cytotoxic concentrations. The immunofluorescence staining revealed that Extract I at concentrations of 3 μg/mL and 6 μg/mL can inhibit the protein expression levels of NF-κB and MMP-3 induced by IL-1β in SW 1353 cells.

Hence, Zingiber zerumbet rhizome extracts and extract composites are believed to exhibit anti-oxidative effects and anti-inflammatory effects, in both the immune cell model and chondrocyte model.

### Example 10—Evaluation of Effect on Relieving Osteoarthritis in Animal Model

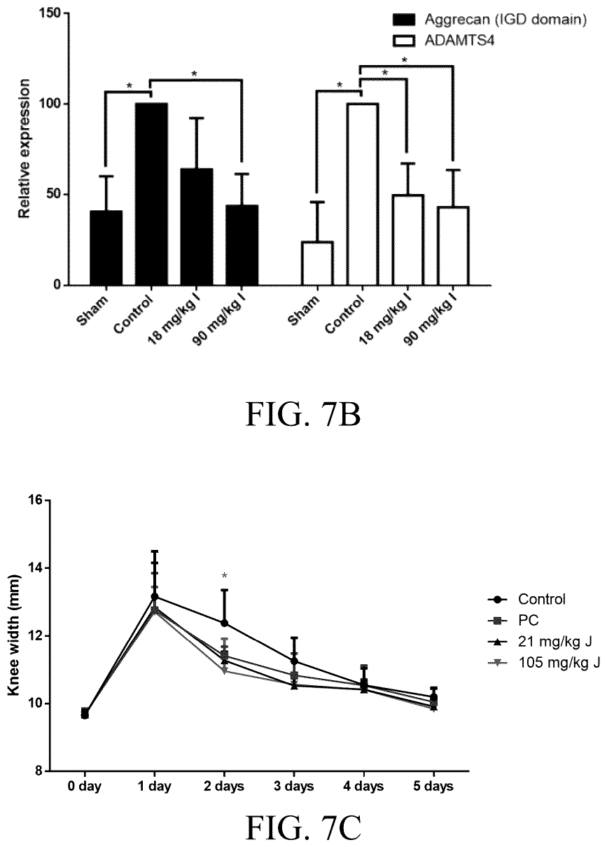

Wistar rats were injected with 50 μL of monosodium iodoacetate (MIA, 80 mg/mL in PBS) at day 0 into the right knee to induce osteoarthritis in the control group and treated groups, and the non-injected left knee was used as the reference. Rats in the sham group were injected the equal volume of PBS to the right knee. Rats were fed with Extract I (18 mg/kg or 90 mg/kg) daily for 10 days, and weight-bearing ratios (the ratio of weight on the right leg to the left leg) were measured on day 4, 7, and 10. Rats were fed with Extract J (21 mg/kg or 105 mg/kg) daily for 14 days, and weight-bearing ratios and paw-lick latency time (sec) were measured on day 7 and 14. On Day 4, the weight-bearing ratios of rats in the tested group of Extract I showed no significantly different compared to the control (p>0.05). On Day 7, both of the group administered with Extract I at 18 mg/kg and 90 mg/kg showed to elevate and recover the weight-bearing ratios, and had significant differences compared to the control (p<0.05). On Day 10, while the weight-bearing ratio of the tested groups of Extract I continued to rise, a significant difference was observed only in the comparison between the control group and the group administered with Extract I at 90 mg/kg (see Table 6). On Day 10, the rats were sacrificed, their serum was analyzed for PGE₂ concentration, and the protein expression in their joint menisci were analyzed. The results showed that the PGE₂ levels in the serum of rats fed with Extract I significantly decreased in a dose-dependent manner (see, FIG. 7A), and the protein expression of ADAMTS4 and aggrecan in cartilage is also significantly reduced (see, FIG. 7B).

TABLE 6
The ratio of weight bearing (R/L)

| | 0 day | 4 days | 7 days | 10 days |
|---|---|---|---|---|
| Sham | 1.02 ± 0.06 | 0.99 ± 0.02* | 0.99 ± 0.02* | 1.09 ± 0.08* |
| Control | 1.01 ± 0.05 | 0.42 ± 0.16 | 0.46 ± 0.11 | 0.61 ± 0.09 |
| Extract I, 18 mg/kg | 1.00 ± 0.03 | 0.52 ± 0.23 | 0.66 ± 0.11* | 0.68 ± 0.08 |
| Extract I, 90 mg/kg | 0.99 ± 0.04 | 0.64 ± 0.08 | 0.74 ± 0.05* | 0.79 ± 0.03* |

*p < 0.05, compared with the control

Extract J was also used in the same animal model, at concentrations of 21 mg/kg and 105 mg/kg. After feeding with Extract J or Indomethacin (the positive control; PC, 2 mg/kg) for two weeks, the weight-bearing ratios in the groups fed with Extract J at a concentration of 21 mg/kg or 105 mg/kg, and positive control were recovered and had significant differences compared to the control (p<0.05, see Table 7). The paw-lick latency time can be increased in the group administered with 105 mg/kg of Extract J or positive control by analgesic activities (see Table 8). Further, swelling caused by knee inflammation with MIA induction can be significantly relieved in the group administered with 105 mg/kg of Extract J (on Day 2, see FIG. 7C).

TABLE 7
The ratio of weight bearing (R/L)

| | 0 week | 1 week | 2 weeks |
|---|---|---|---|
| Control | 1.02 ± 0.03 | 0.62 ± 0.06 | 0.52 ± 0.11 |
| Indomethacin (2 mg/kg) | 1.03 ± 0.04 | 0.71 ± 0.11 | 0.66 ± 0.04* |
| Extract J, 21 mg/kg | 1.00 ± 0.03 | 0.65 ± 0.16 | 0.68 ± 0.05* |
| Extract J, 105 mg/kg | 1.01 ± 0.04 | 0.71 ± 0.05 | 0.78 ± 0.05* |

*p < 0.05, compared with the control

TABLE 8
Paw-lick latency time (s)

| | 0 week | 1 week | 2 weeks |
|---|---|---|---|
| Control | 17.47 ± 5.27 | 10.07 ± 3.62 | 15.00 ± 3.25 |
| Indomethacin (2 mg/kg) | 16.02 ± 2.25 | 13.93 ± 3.06 | 23.29 ± 4.43* |
| Extract J, 21 mg/kg | 18.31 ± 1.74 | 16.05 ± 4.13 | 16.59 ± 4.86 |
| Extract J, 105 mg/kg | 17.92 ± 5.29 | 18.43 ± 6.36 * | 22.09 ± 2.98* |

*p < 0.05, compared with the control

Rats were fed with RAW (powdered Zingiber zerumbet rhizome, 387 mg/kg), Extract composite (I+M=2:8, IM2, 96 mg/kg), and Extract I (18 mg/kg) daily for 10 days, which were used at a dosage equivalent to zerumbone of 12 mg/kg. The weight-bearing ratios (the ratio of weight on the right leg to the left leg) were measured on day 3 and day 7, and the results are shown in Table 9. On the Day 3 and Day 7, the weight-bearing ratio of rats fed with RAW (387 mg/kg), Extract composite IM2 (I+M=2:8, 96 mg/kg), Extract I (18 mg/kg), and positive control showed significantly better than that of the control group. On the day 7, the weight-bearing ratio of rats fed with Extract composite IM2 (I+M=2:8, 96 mg/kg), Extract I (18 mg/kg), and positive control showed significantly better than that of the RAW (387 mg/kg) group.

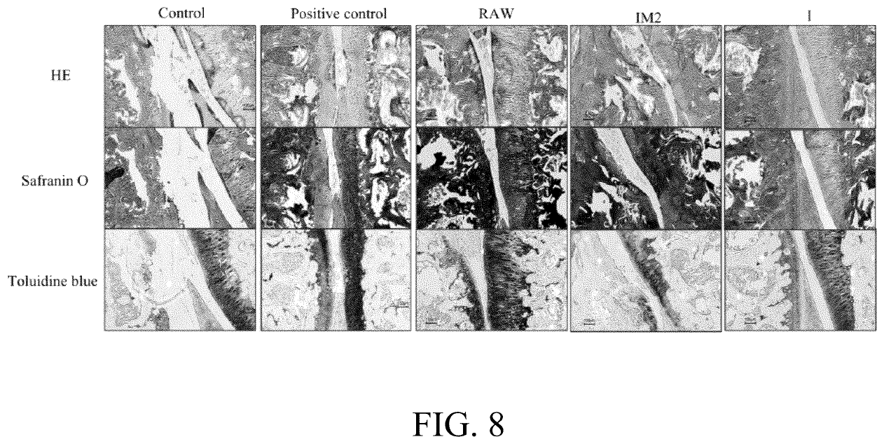

Additionally, femoral joint tissues were collected for pathological sectioning and immunostaining, and the results are shown in FIG. 8. H&E staining patterns revealed that the cartilage layer surface in the control group was uneven and had fracture surfaces, while the groups fed with Extract I showed recovery of femoral joint tissues. Toluidine Blue staining can indicate chondroitin sulfate in the cartilage (as blue), and the results showed that after feeding with Extract I, the glycoprotein content in the cartilage was higher than that of the control group.

TABLE 9
The ratio of weight bearing (R/L)

| | 0 day | 3 days | 7 days |
|---|---|---|---|
| Control | 0.99 ± 0.06 | 0.49 ± 0.06 | 0.40 ± 0.05 |
| Indomethacin (2 mg/kg) | 0.92 ± 0.09 | 0.76 ± 0.07* | 0.80 ± 0.08*^{#} |
| RAW (387 mg/kg) | 1.00 ± 0.03 | 0.67 ± 0.04* | 0.67 ± 0.01* |
| Extract composite IM2 (96 mg/kg) | 1.01 ± 0.00 | 0.67 ± 0.07* | 0.70 ± 0.07* |
| Extract I (18 mg/kg) | 1.03 ± 0.04 | 0.76 ± 0.01* | 0.79 ± 0.05*^{#} |

*p < 0.05, compared with the control;
^{#}p < 0.05, compared with the RAW

Given the above, Zingiber zerumbet extracts and extract composites may exhibit anti-oxidative effects, anti-inflammatory effects, neuroprotection, promotion of synapse (out) growth, improvement in cognitive dysfunctions (in particular in neurodegenerative diseases), prevention and/or treatment of osteoarthritis, etc., in a subject and preferably these effects are significantly better than those exhibited by the raw materials of Zingiber zerumbet. The extracts and extract composites may also provide bioavailability similar to that of a direct administration of zerumbone.

A person of ordinary skill in the art of the subject invention should understand that variations and modifications may be made to the teaching and the disclosure of the subject invention without departing from the spirit and scope of the subject application. Based on the contents above, the subject application intends to cover any variations and modifications thereof with the proviso that the variations or modifications or their equivalents fall within the scope as defined in the appended claims.

We claim:

1. A Zingiber zerumbet rhizome extract or extract composite, characterized in comprising at least 120 mg/g zerumbone/extract and at least 5.0 mg/g 3-O-methyl kaempferol/extract.

2. The extract or extract composite according to claim 1, wherein the ratio of 3-O-methyl kaempferol to zerumbone is at least 0.05.

3. The extract or extract composite according to claim 1, which comprises (a) a supercritical fluid CO₂ extract of Zingiber zerumbet rhizome and (b) an ethanolic extract of the residue of the supercritical fluid CO₂ extract of Zingiber zerumbet rhizome.

4. The extract or extract composite according to claim 1, which comprises (a) an oil phase or components of water vapor extract of Zingiber zerumbet rhizome and (b) an ethanolic extract of the residue of the water vapor extract of Zingiber zerumbet rhizome.

5. The extract or extract composite according to claim 3, wherein the ethanolic extract is obtained by extraction with 75% ethanolic solution.

6. The extract or extract composite according to claim 4, wherein the ethanolic extract is obtained by extraction with 75% ethanolic solution.

7. The extract or extract composite according to claim 1, characterized in further comprising at least one component selected from the group consisting of α-humulene, β-eudesmol and caryophyllene oxide.

8. The extract or extract composite according to claim 7, characterized in further comprising at least one component selected from the group consisting of camphene and eucalyptol.

9. The extract or extract composite according to claim 3, characterized in further comprising at least one component selected from the group consisting of zerumbone epoxide and kaempferol-3-O-(3,4-di-O-acetyl-α-L-rhamnopyranoside).

10. The extract or extract composite according to claim 7, wherein (i) the weight ratio of α-humulene to zerumbone is 0.062+/−10%, (ii) the weight ratio of β-eudesmol to zerumbone is 0.016+/−10%, and/or (iii) the weight ratio of caryophyllene oxide to zerumbone is 0.073+/−10%.

11. The extract or extract composite according to claim 8, wherein (i) the weight ratio of α-humulene to zerumbone is 0.126+/−10%, (ii) the weight ratio of β-eudesmol to zerumbone is 0.025+/−10%, (iii) the weight ratio of caryophyllene oxide to zerumbone is 0.107+/−10%, (iv) the weight ratio of camphene to zerumbone is 0.078+/−10%, and/or (v) the weight ratio of eucalyptol to zerumbone is 0.027+/−10%.

12. A method of improving cognitive dysfunctions in neurodegenerative diseases in a subject, comprising administering an effective amount of Zingiber zerumbet rhizome extract or extract composite according to claim 1 to the subject.

13. A method of improving cognitive dysfunctions in neurodegenerative diseases in a subject, comprising administering an effective amount of Zingiber zerumbet rhizome extract or extract composite according to claim 1 to the subject.

14. The method according to claim 13, wherein the ratio of 3-O-methyl kaempferol to zerumbone is at least 0.05.

15. The method according to claim 13, wherein the cognitive dysfunctions comprise age-associated memory impairment, age-associated cognitive decline or mild cognitive impairment.

16. The method according to claim 13, wherein the neurodegenerative diseases include Parkinson's disease (PD), Alzheimer's disease (AD), Huntington's disease (HD), dementia, prior diseases, motor neuron disease, spinocerebellar Ataxia (SCA), amyotrophic lateral sclerosis (ALS) or Lewy body diseases.

17. A method of preventing and/or treating osteoarthritis in a subject, comprising administering an effective amount of Zingiber zerumbet rhizome extract or extract composite according to claim 1 to the subject.

18. The method according to claim 17, wherein osteoarthritis is primary osteoarthritis, occupational osteoarthritis and/or secondary osteoarthritis.
